(19) United States
(12) Patent Application Publication
(10) Pub. No.: US 2024/0042189 A1
(43) Pub. Date: Feb. 8, 2024

(54) Container With Threaded Connector

(71) Applicant: Weiler Engineering Inc., Elgin, IL (US)

(72) Inventors: Valentin Cosman, Barrington Hills, IL (US); Andrew W. Goll, Huntley, IL (US); Christopher B. Isaac, Algonquin, IL (US)

(21) Appl. No.: 18/485,871

(22) Filed: Oct. 12, 2023

Related U.S. Application Data
(63) Continuation-in-part of application No. 17/706,022, filed on Mar. 28, 2022.

(51) Int. Cl. A61M 39/20

(52) U.S. Cl. CPC A61M 39/20; A61M 2039/1033

(57) ABSTRACT

A hermetically sealed thermoplastic container or ampoule having a hollow body portion, a neck portion, unitary with the body portion and terminating in a circumferential flat sealing surface that defines an access aperture, and a removable cap. The neck portion is provided with an external thread and defines an access passageway to the hollow body portion. Part of the neck portion is configured as a tapered female connector adapted to receive a tapered male connector when the access passageway is open.

### CROSS-REFERENCE TO RELATED APPLICATION

This application is a continuation-in-part of U.S. Ser. No. 17/706,022 filed on Mar. 28, 2022, which is incorporated herein by reference in its entirety.

### FIELD OF INVENTION

This invention relates to a hermetically sealed thermoplastic container or ampoule with tapered connector provided with external threads.

### BACKGROUND OF THE INVENTION

Hermetically sealed containers manufactured by the so-called blow-fill-seal method have enjoyed widespread acceptance for dispensing liquid medicaments and the like.

Tapered connectors or fittings are used for making leak-free connections between a male-taper fitting and its mating female part on various containers for medications, medical and laboratory instruments, including hypodermic syringe tips and needles, and the like. There are two varieties of taper connectors: lock connectors and slip connectors. Lock connectors have tapered male and female fittings securely joined to one another by means of a tabbed hub on the female fitting which engages internal threads in a collar or sleeve on the male fitting. Slip connectors, sometimes referred to as slip tip fittings, are pressed together and held to one another by friction. Such taper connectors, however, do not always provide an adequate seal at the connecting site.

It would be desirable to provide a hermetically sealed thermoplastic container or ampoule with an easily removable cap or closure and with an access passageway sized to receive a variety of implements with tapered connectors, such as those specified in International Standard ISO 80369, having an enhanced sealing capacity. The present invention provides such a container.

### SUMMARY OF THE INVENTION

A hermetically sealed container or ampoule is made of a thermoplastic material, such as a polyolefin, and the like, and is provided with a readily removable cap which occludes an access passageway defined by a neck portion of the container. The access passageway usually is aligned with the longitudinal axis of the container or ampoule.

A container or ampoule embodying the present invention comprises a hermetically sealed container of a thermoplastic material having a hollow body portion, a neck portion unitary with the body portion and which terminates in a circumferential, annular, flat sealing surface which defines an access aperture or opening sized to receive a tapered male connector having internal threads. A removable cap is joined to the neck portion by a frangible web unitary with the removable cap and with the neck portion. The neck portion of the container also defines an access passageway to the hollow body portion. A continuous external thread is provided on the neck portion. The passageway is configured as a tapered female connector sized to receive a tapered male connector when the cap is removed and the access passageway is open. The annular, flat sealing surface has an area that is at least 1.85 times the area of the access aperture.

Part of the neck portion is configured as a tapered female connector or fitting adapted to receive a tapered male connector or fitting when the access passageway is open. The neck portion around its periphery includes a continuous external thread that urges the external face of a tapered male connector or fitting against a tapered wall portion of the access passageway. The threads enhance sealing and also contribute to rigidity and dimensional stability of the access passageway. The continuous thread preferably is a buttress thread, more preferably an “M” style modified buttress thread.

The removable cap unitary with the dispensing nozzle occludes the access passageway. The removable cap is connected at its rim to the neck portion by a frangible web that is unitary with the cap as well as with the flat sealing surface that surrounds the access passageway.

The removable cap has an elongated tab or trigger that is unitary with the cap and extends across at least a major part of the top surface of the cap. The tab is positioned across the top surface of the removable cap for engagement therewith when the tab or trigger is manipulated either in a twisting motion or generally about the longitudinal axis of the nozzle. Manipulation of the tab severs the frangible web and permits removal of the cap to gain access to the container or ampoule contents.

### BRIEF DESCRIPTION OF THE DRAWINGS

In the drawings,

FIG. 1 is a fragmentary front elevational view, partly in section, showing a hermetically sealed container embodying the present invention and having a removable cap in place;

FIG. 2 shows a fragmentary front elevational view of the container of FIG. 1 without the removable cap and with external threads on the neck portion of the container;

FIG. 3 is a fragmentary front elevation view, partly in section, and with access aperture exposed;

FIG. 4 is a fragmentary front elevational view, partly in section, showing a tapered male Luer lock connector with a fixed collar partially received in a tapered female connector of a container embodying the present invention and having a neck portion provided with external threads;

FIG. 5 shows the container of FIG. 4 with the tapered male Luer lock connector fully engaged with the tapered female connector;

### DETAILED DESCRIPTION OF THE PREFERRED EMBODIMENTS

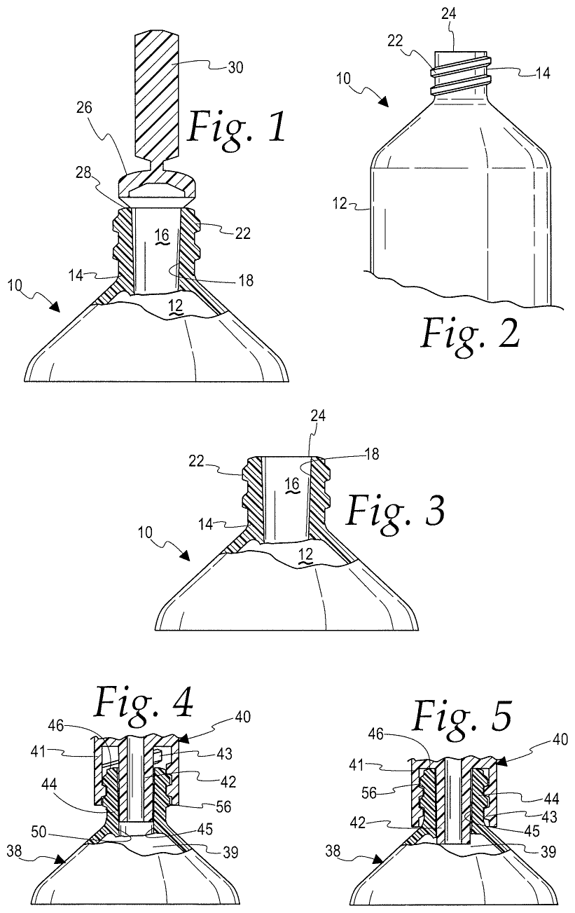

Referring to the drawings, FIGS. 1, 2, and 3 show a portion of a hermetically sealed container 10 made of a thermoplastic material. Container 10 has a hollow body portion 12 and a neck portion 14 unitary with body portion 12. Neck portion 14 defines access passageway 16 which has a tapered throat region 18. Access passageway 16 terminates in a circumferential, annular, flat sealing surface 24 which defines an opening sized to receive the tip of a tapered male connector. The outer diameter-to-inner diameter ratio of the annular, flat sealing surface 24 is at least 1.6, preferably in the range of 1.6 to about 1.7. Removable cap 26, when in place, occludes access passageway 16 and is connected to neck portion 14 by a unitary frangible web 28. Removable cap 26 is provided with optional tab 30 which is unitary with cap 26. Manipulation of tab 30 severs frangible web 28 and provides access to passageway 16 as will be discussed in greater detail below. Continuous external buttress threads 22 are provided on neck portion 14, sized to engage internal threads of a collar that surrounds a tapered tip of a tapered male connector.

The taper angle on the tapered throat region 18 varies to match the taper angle of a tapered male connector to be connected thereto. The taper angle preferably is in the range of about 5 degrees to about 6 degrees so as to comply with International Standard ISO 80369-6 for small bore connectors for liquids and gases in healthcare applications.

FIGS. 4 and 5 show a tapered Luer lock male connector 40 on container 38 having a hollow body portion 39 and embodying the present invention. Neck portion 44 of container 38 defines tapered access passageway 45 and flat sealing surface 46. Access passageway 45 has tapered throat region 50 sized to receive tip 42 of tapered male connector 40. External threads 56 are provided on neck portion 44. Tapered male connector has a cylindrical collar 41 provided with internal threads 43. As tapered male connector 40 is threaded onto external threads 56, tip 42 enters tapered throat region 50 of neck portion 44 connector and is urged against the wall of the tapered portion of access passageway 45 and against flat sealing surface 46 as shown in FIG. 4 and FIG. 5, thereby providing an effective, positive seal for the tapered male connector 40 when the tapered portion or a tip thereof is seated in tapered throat region 50.

The specific external thread type provided on the neck portion of the container can vary and is determined by the type of male connector engaging the neck portion. Right-handed buttress threads are preferred for this purpose, more preferably, “M” style modified buttress threads.

Specifications for the external thread can vary depending on intended use. To engage a male Luer lock connector, the external thread is compliant with International Standard ISO 80369-7. To engage a small bore male Luer lock connector, the external thread is compliant with International Standard ISO 80369-6.

The procedure for forming, filling and sealing of thermoplastic, hermetically sealed containers is well known in the art and is generally described in U.S. Pat. No. 3,597,793 to Weiler et al. Containers embodying the present invention can be formed, filled, and sealed under sterile or aseptic conditions using techniques known in the art as described in U.S. Pat. No. 4,178,976 to Weiler et al. Suitable thermoplastic polyolefin materials of construction are polypropylene (PP), high density polyethylene (HDPE), low density polyethylene (LDPE), and the like. A particularly preferred material of construction is LDPE.

An illustrative, sealed thermoplastic container embodying the present invention has a volume of about 20 milliliters and a neck portion sealed by a unitary removable cap joined to the neck portion by a frangible web. The neck portion defines an access aperture and an access passageway. The access aperture is surrounded by an annular, flat sealing surface which becomes exposed upon removal of the cap.

The annular, flat sealing surface has an outer diameter of about 0.283 inches and an inner diameter of about 0.17 inches which is also the diameter of the access aperture. The sealing surface has an exposed area of about 0.042 square inches, about 1.851 times the area of the access aperture for contacting a syringe tip received into the access aperture.

The tapered access passageway has a taper of about 5 degrees and a length of about inches, i.e., about 1.735 times the diameter of the access aperture.

The neck portion is surrounded by a right-hand “M” style modified buttress thread.

The foregoing description and the drawings are illustrative of the invention, but are not to be taken as limiting. Still other variants within the spirit and scope of this invention are possible and will readily present themselves to those skilled in the art.

1. A hermetically sealed container of a thermoplastic material and comprising a hollow body portion, a neck portion unitary with the hollow body portion, and a removable cap joined to the neck portion by a frangible web unitary with the removable cap and with the neck portion;
the neck portion terminating in a circumferential, annular, flat sealing surface which defines an access aperture sized to receive a tapered male connector having internal threads, defining a tapered access passageway to the hollow body portion, and provided with a continuous external thread;
the ring-like, flat sealing surface having an area at least 1.85 times larger than an area defined by the access aperture; and
the access passageway extending continuously from the ring-like, flat sealing surface to the hollow body portion and configured as a tapered female connector sized to receive said tapered male connector.

2. The hermetically sealed container in accordance with claim 1 wherein the ring-like, flat sealing surface has an outer diameter-to-inner diameter ratio in the range of 1.6 to 1.7.

3. The hermetically sealed container in accordance with claim 1 wherein the said continuous external thread is a buttress thread.

4. The hermetically sealed container in accordance with claim 3 wherein said buttress thread is an “M” style modified buttress thread.
